(19) United States
(12) Patent Application Publication
(10) Pub. No.: US 2021/0343031 A1
(43) Pub. Date: Nov. 4, 2021

(54) LESION LOCALIZATION IN AN ORGAN

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Yanling CHI, Singapore (SG); Weimin HUANG, Singapore (SG); Jiayin ZHOU, Singapore (SG); Kyaw Kyar TOE, Singapore (SG); Stephen CHANG, Singapore (SG); Chee Kong CHUI, Singapore (SG)

(21) Appl. No.: 17/272,152

(22) PCT Filed: Aug. 29, 2018
(86) PCT No.: PCT/SG2018/050437

§ 371 Date: Feb. 26, 2021

(51) Int. Cl. G06T 7/33; G06T 7/73; G06T 3/60; A61B 18/14

(52) U.S. Cl. CPC G06T 7/33; G06T 7/73; G06T 3/60; A61B 18/1492; A61B 2018/00994; G06T 2207/10081; G06T 2207/10132; G06T 2207/30204; G06T 2207/30096

(57) ABSTRACT

The invention relates to a computerized method (200) for localizing a lesion in an organ of a subject, comprises performing: a first image registration operation (400) for determining a rigid transformation matrix based on alignment of a two-dimensional ultrasound (2D-US) image representation (116) and a three-dimensional computed tomography (3D-CT) image representation (120) of the organ, the 2D-US image representation (116) acquired from a transducer probe (114); a second image registration operation (500) for refining the rigid transformation matrix based on image feature descriptors of the 2D-US and 3D-CT image representations (116, 120); and a localization operation (600) for localizing the lesion relative to the transducer probe (114) based on the refined rigid transformation matrix and a 3D-CT position of the lesion in the 3D-CT image representation (120). A system for performing the method is also disclosed herein. The system may further comprise an ablation apparatus for radio frequency ablation of the lesion.

### TECHNICAL FIELD

The present disclosure generally relates to lesion localization in an organ. More particularly, the present disclosure describes various embodiments of a computerized method and a system for localizing a lesion in an organ of a subject, such as a tumor in a liver of a person, using ultrasound and computed tomography image representations of the organ.

### BACKGROUND

Liver cancer is the sixth most common cancer worldwide and some statistics indicate that there are approximately 782,000 new cases diagnosed globally in 2012. Surgical resection of liver tumors is considered as the gold standard for treatment. About 20% of patients diagnosed with liver cancer are suitable for open surgery. An alternative treatment for the other patients is ultrasound (US) guided radiofrequency ablation (RFA). Because an ablation size is relatively small, multiple applications of RF waves are required for ablating the liver tumor. However, gas bubbles or bleeding resulting from the initial applications may reduce visibility of the liver tumor on US images subsequently, thereby decreasing the ablation efficacy.

Image fusion or registration of intra-intervention US images with pre-intervention computed tomography (CT) images is able to improve tumor localization during RFA. However, there are difficulties due to respiration, pose position, and similarity measurements of US and CT images. US and CT have different principles of imaging, which results in difference appearances of the same organ. In addition, the field of view of US images is limited and an US image usually acquires little details within the liver, while a CT image is more detailed.

Reference [16] describes image fusion of three-dimensional (3D) US images with 3D-CT images. The 3D-US images may be acquired using a 3D-US scanner, reconstructed from a series of two-dimensional (2D) US scans, or simulated from the 3D-CT image. However, 3D-US scanners are not widely available in hospitals or other medical facilities. Moreover, 3D-US simulation and reconstruction are complicated, time consuming, and prone to errors introduced by clinicians. The use of 3D-US images thus presents challenges in localization of liver tumors.

Therefore, in order to address or alleviate at least one of the aforementioned problems and/or disadvantages, there is a need to provide an improved system and computerized method for localizing a lesion in an organ of a subject using US and CT image representations of the organ.

### SUMMARY

According to an aspect of the present disclosure, there is a system and computerized method for localizing a lesion in an organ of a subject. The system comprises a transducer probe for acquiring a two-dimensional ultrasound (2D-US) image representation of the organ; and a computer device communicable with the transducer probe. The computer device comprises an image registration module and a localization module configured for performing steps of the method. The method comprises performing: a first image registration operation for determining a rigid transformation matrix based on alignment of the 2D-US image representation and a three-dimensional computed tomography (3D-CT) image representation of the organ, the 2D-US image representation acquired from the transducer probe; a second image registration operation for refining the rigid transformation matrix based on image feature descriptors of the 2D-US and 3D-CT image representations; and a localization operation for localizing the lesion relative to the transducer probe based on the refined rigid transformation matrix and a 3D-CT position of the lesion in the 3D-CT image representation.

An advantage of the present disclosure is that localization of the lesion in the organ is improved by using the 2D-US and 3D-CT image representations and refinements to the rigid transformation matrix. The localization may be performed in collaboration with an image-guided intervention procedure such as radiofrequency ablation to target the lesion for more effective ablation.

A system and computerized method for localizing a lesion in an organ of a subject using US and CT image representations of the organ according to the present disclosure are thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1 is a schematic illustration of a system for localizing a lesion in an organ of a subject.

FIG. 2 is a flowchart illustration of a computerized method for localizing a lesion in an organ of a subject.

FIG. 3 is a flowchart illustration of a calibration operation of the method.

FIG. 4A is a flowchart illustration of a first image registration operation of the method.

FIG. 4B is an illustration of a 2D-US image representation.

FIG. 4C is an illustration of a first aligned image representation from fiducial-based alignment of the 2D-US and 3D-CT image representations.

FIG. 5A is a flowchart illustration of a second image registration operation of the method.

FIG. 5B is an illustration of a second aligned image representation from feature-based alignment of the 2D-US and 3D-CT image representations.

FIG. 5C illustrates multi-modal similarity metrics of the 2D-US and 3D-CT image representations.

FIG. 6 is a flowchart illustration of a localization operation of the method.

FIG. 7 is an illustration of a pseudocode of the method.

FIG. 8A are illustrations of the 2D-US and 3D-CT image representations and multi-modal similarity metrics for an inhalation phase.

FIG. 8B are illustrations of the 2D-US and 3D-CT image representations and multi-modal similarity metrics for an exhalation phase.

FIG. 9 is an illustration of a performance comparison table of the method against other known methods.

FIG. 10 is an illustration of an ablation apparatus.

### DETAILED DESCRIPTION

In the present disclosure, depiction of a given element or consideration or use of a particular element number in a particular figure or a reference thereto in corresponding descriptive material can encompass the same, an equivalent, or an analogous element or element number identified in another figure or descriptive material associated therewith. The use of “I” herein, in a figure, or in associated text is understood to mean “and/or” unless otherwise indicated. The recitation of a particular numerical value or value range herein is understood to include or be a recitation of an approximate numerical value or value range.

For purposes of brevity and clarity, descriptions of embodiments of the present disclosure are directed to a system and computerized method for localizing a lesion in an organ of a subject using ultrasound and computed tomography image representations of the organ, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, known systems, methods, procedures, and components have not been described in detail so as to not unnecessarily obscure aspects of the embodiments of the present disclosure.

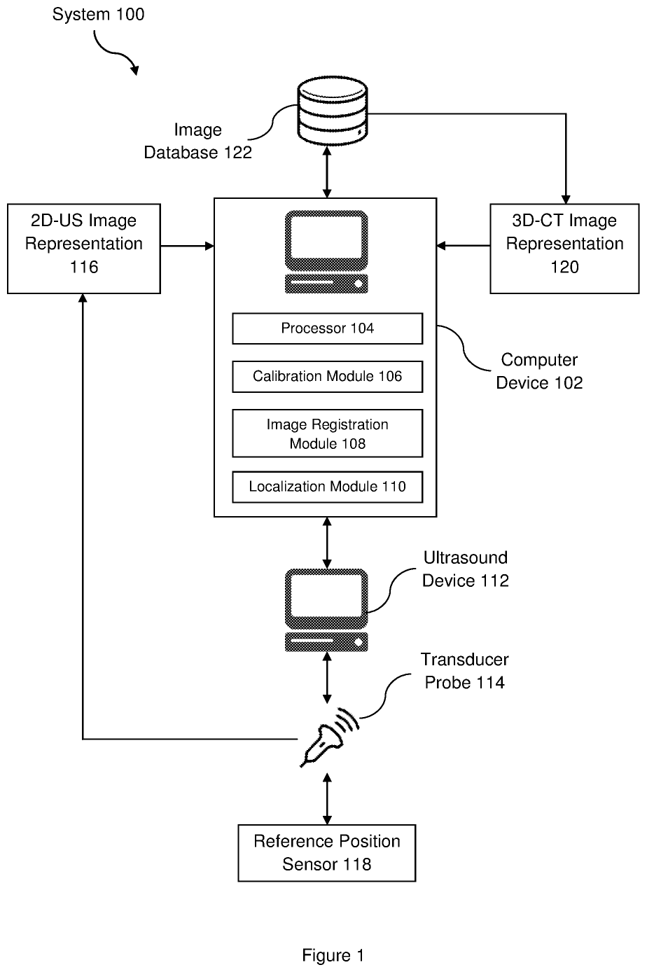

In representative or exemplary embodiments of the present disclosure with reference to FIG. 1, there is a system 100 configured for performing a computerized or computer-implemented method 200 for localizing a lesion in an organ of a subject. Specifically, the system 100 includes a computer device 102 having a processor 104 and various components/modules, including a calibration module 106, an image registration module 108, and a localization module 110, configured for performing the method 200.

As used herein, the terms component and module are generally intended to refer to a computer-related entity, either hardware, a combination of hardware and software, software, or software in execution. For example, a component or a module may be, but is not limited to being, a process running on a processor, a processor, an object, an executable, a thread of execution, a program, and/or a computer. Additionally, the processor 104 and the modules 106, 108, and 110 are configured for performing various operations/steps of the method 200 and are configured as part of the processor 104. Each module 106/108/110 includes suitable logic/algorithm for performing various operations/steps of the method 200. Such operations/steps are performed in response to non-transitory instructions operative or executed by the processor 104.

The system 100 further includes an ultrasound (US) device 112 and a transducer probe 114 connected thereto for acquiring an US image representation of the organ. The computer device 102 is communicatively connected to or communicable with the US device 112 and transducer probe 114 for receiving the US image representation acquired from the transducer probe 114 used on the subject. Specifically, the US device 112 and transducer probe 114 are configured for acquiring a two-dimensional ultrasound (2D-US) image representation 116 of the organ including the lesion.

The system 100 further includes a reference position sensor 118 disposed on the transducer probe 114, specifically at an end thereof. The reference position sensor 118 is calibrated for localizing the lesion and determining the position of the lesion relative to the transducer probe 114/reference position sensor 118.

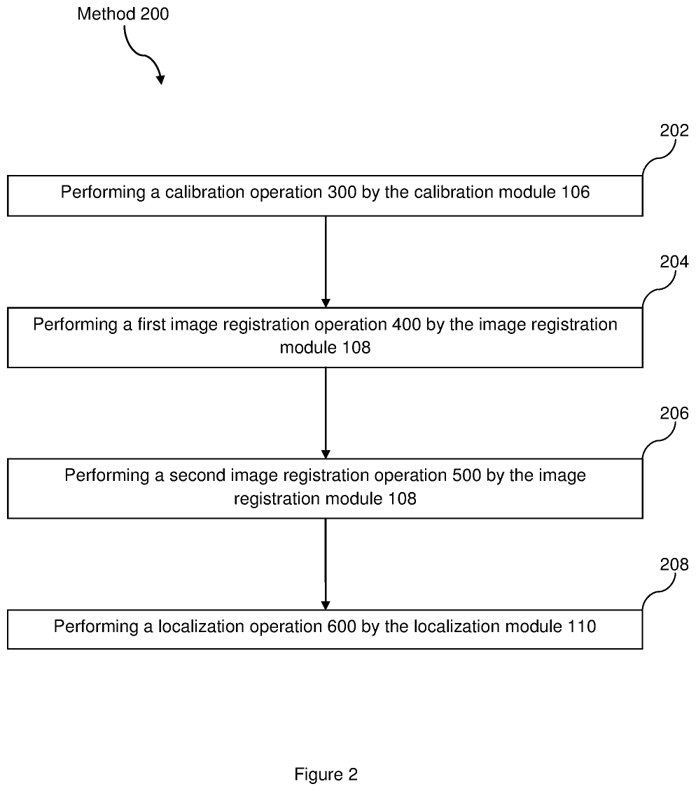

Further with reference to FIG. 2, the method 200 for localizing a lesion in an organ of a subject includes a number of stages. Specifically, the method 200 includes an optional calibration stage 202 of performing a calibration operation 300 by the calibration module 106, a first stage 204 of performing a first image registration operation 400 by the image registration module 108, a second stage 206 of performing a second image registration operation 500 by the image registration module 108, and a third stage 208 of performing a localization operation 600 by the localization module 110.

The subject may be a person or an animal, such as a pig or swine. As used herein, a lesion is defined as a region in an organ which has suffered damage through injury or disease. Non-limiting examples of a lesion include a wound, ulcer, abscess, and tumor. Lesions, specifically tumors, may be present in organs such as lungs, kidneys, and livers. In some embodiments, the method 200 is performed by the system 100 for localizing of a tumor in a liver of a pig or swine.

In some embodiments, the method 200 includes the calibration stage 202. The calibration operation 300 is performed by the calibration module 106 of the computer device 102 for calibrating the transducer probe 114. Specifically, the calibration operation 300 includes defining a reference coordinate frame of the transducer probe 114, wherein the lesion is localized in the reference coordinate frame. In some other embodiments, the transducer probe 114 has been pre-calibrated before the method 200 is performed for localizing the lesion relative to the transducer probe 114.

In the first stage 204, the first image registration operation 400 is performed by the image registration module 108 of the computer device 102 for determining a rigid transformation matrix based on alignment of the 2D-US image representation 116 and a three-dimensional computed tomography (3D-CT) image representation 120 of the organ, the 2D-US image representation 116 acquired from the transducer probe 114. In the second stage 206, the second image registration operation 500 is performed by the image registration module 108 for refining the rigid transformation matrix based on image feature descriptors of the 2D-US image representation 116 and 3D-CT image representation 120. In the third stage 208, the localization operation 600 is performed by the localization module 110 of the computer device 102 for localizing the lesion relative to the transducer probe 114 based on the refined rigid transformation matrix and a 3D-CT position of the lesion in the 3D-CT image representation 120.

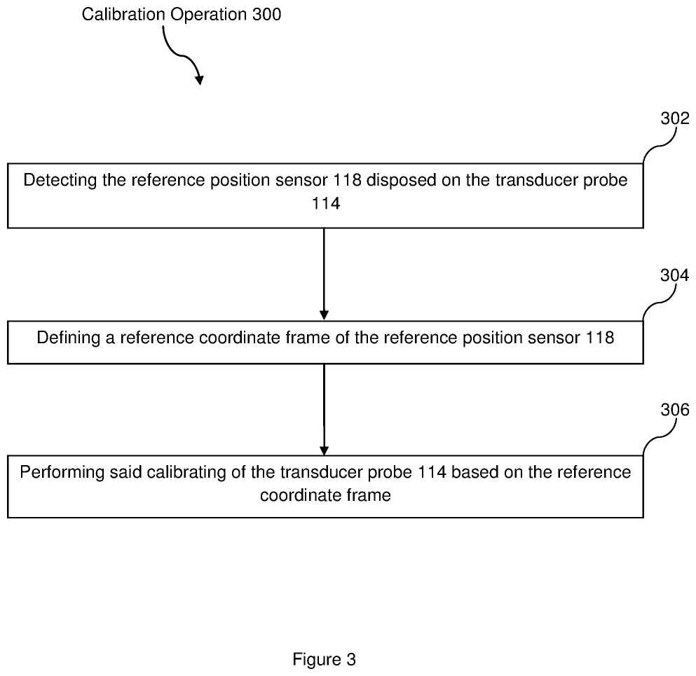

With reference to FIG. 3, the optional calibration operation 300 performed before the first image registration operation 400 includes a step 302 of detecting the reference position sensor 118 disposed on the transducer probe 114. The calibration operation 300 further includes a step 304 of defining a reference coordinate frame of the reference position sensor 118. The calibration operation 300 further includes a step 306 of performing said calibrating of the transducer probe 114 based on the reference coordinate frame.

The reference coordinate frame of the transducer probe 114/reference position sensor 118 includes a reference origin and three reference orthogonal axes to represent a 3D space. As the reference position sensor 118 is disposed on the transducer probe 114, the 2D-US lesion position on the 2D-US image representation 116 can be transformed to the reference coordinate frame, thereby localizing and positioning the lesion in the reference coordinate frame according to the reference origin and three reference orthogonal axes.

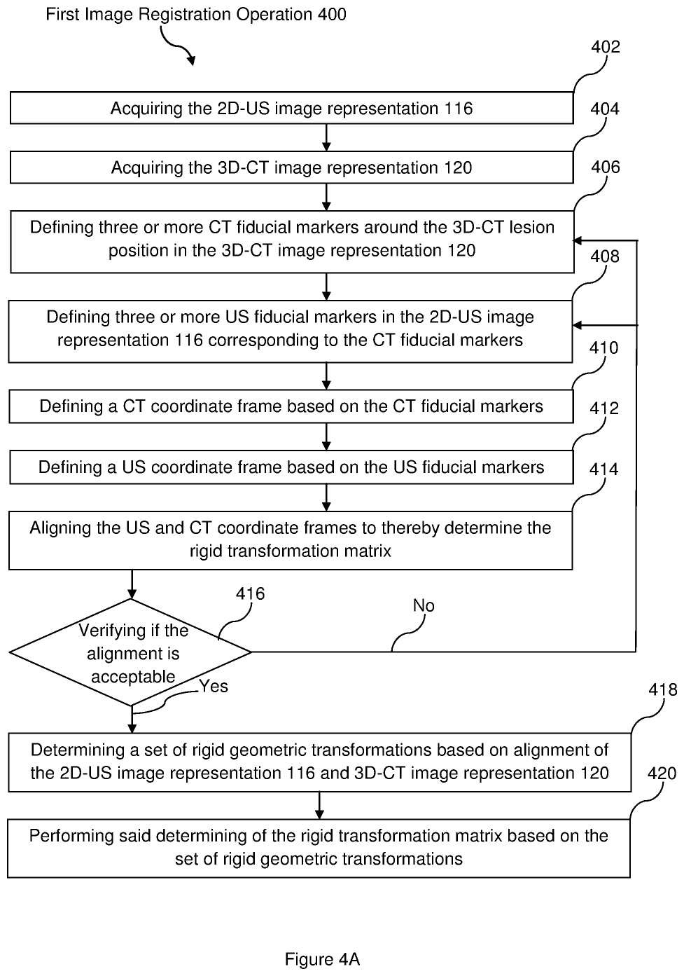

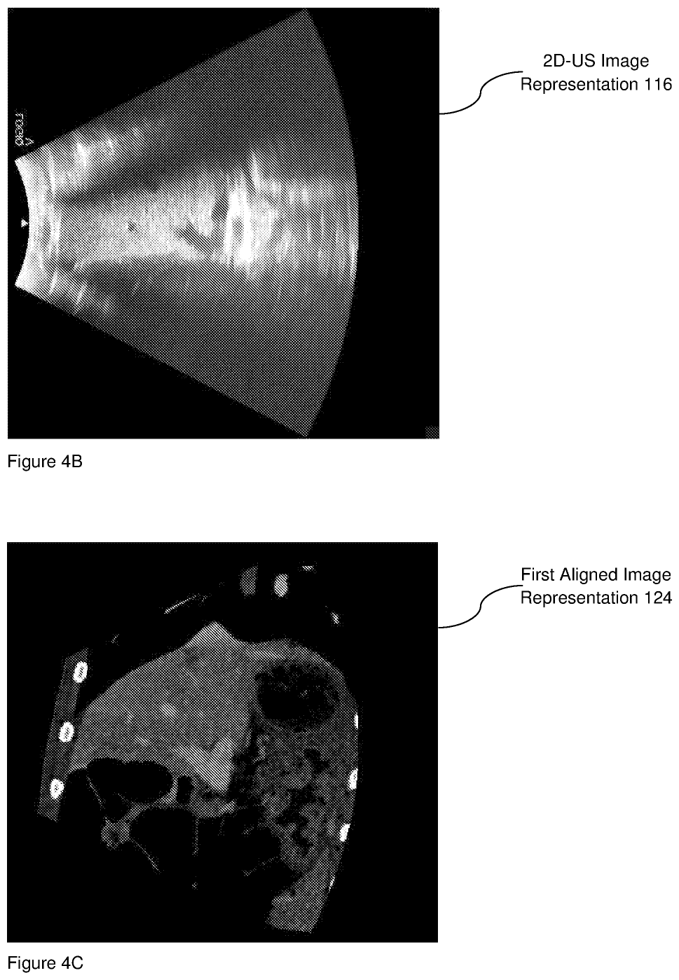

In many embodiments with reference to FIG. 4A, the first image registration operation 400 includes a step 402 of acquiring the 2D-US image representation 116. In one embodiment, the 2D-US image representation 116 is retrieved from an image database 122 storing multiple 2D-US image representations that were pre-acquired from multiple subjects. In another embodiment, the step 402 includes receiving the 2D-US image representation 116 acquired from the transducer probe 114 used on the subject. For example, the 2D-US image representation 116 is acquired from the transducer probe 114 during an intervention procedure to treat the lesion, such as radiofrequency ablation (RFA) which is usually under image guidance such as US images. The intervention procedure may also be referred to as an image-guided intervention (IGI). As the 2D-US image representation 116 is acquired during the RFA for guiding the intervention procedure, the 2D-US image representation 116 may also be referred to as an intra-intervention 2D-US image representation 116. FIG. 4B illustrates an example of the 2D-US image representation 116.

The first image registration operation 400 includes a step 404 of acquiring the 3D-CT image representation 120. Specifically, the step 404 includes retrieving, from the image database 122, the 3D-CT image representation 120 which was pre-acquired from the subject. The 3D-CT image representation 120 was acquired from the subject before the IGI or RFA and stored on the image database 122 and may thus also be referred to as a pre-intervention 3D-CT image representation 120. The image database 122 stores multiple 3D-CT image representations that were pre-acquired from multiple subjects. The image database 122 may reside locally on the computer device 102, or alternatively on a remote or cloud device communicatively linked to the computer device 102. The 3D-CT image representation 120 is an image volume that is collectively formed by multiple 2D-CT image representations or slices which are stacked together. Each 2D-CT image representation has a finite thickness and represents an axial/transverse image of the organ. It will be appreciated that the steps 402 and 404 may be performed in any sequence or simultaneously.

The first image registration operation 400 further includes a step 406 of defining three or more CT fiducial markers around the 3D-CT lesion position in the 3D-CT image representation 120, and a step 408 of defining three or more US fiducial markers in the 2D-US image representation 116 corresponding to the CT fiducial markers. It will be appreciated that the steps 406 and 408 may be performed in any sequence or simultaneously. A fiducial marker is a virtual object, such as a point, placed in the field of view of an imaging or image processing application executed by the computer device 102 for processing the image representations 116 and 120. The US and CT fiducial markers appear in the image representations 116 and 120, respectively, for use as points of reference or measure.

In defining the fiducial markers around the respective lesion positions in the 2D-US image representation 116 and 3D-CT image representation 120, visible anatomical structures are first arbitrarily identified around the lesion. For example, the anatomical structures are vascular tissues that may include vessels and/or vessel bifurcations/junctions/corners where they can be more easily identified, such as the portal vein and portal vein bifurcations. Accordingly, the fiducial markers mark the vascular tissues around the lesion.

The first image registration operation 400 further includes a step 410 of defining a CT coordinate frame based on the CT fiducial markers, and a step 412 of defining a US coordinate frame based on the US fiducial markers. It will be appreciated that the steps 410 and 412 may be performed in any sequence or simultaneously. Each of the US and CT coordinate frames is a plane that passes through the respective US and CT fiducial markers. A plane can be defined by at least three non-collinear points. In one embodiment, there are three US fiducial markers and three CT fiducial markers corresponding in positions to the US fiducial markers. The US coordinate frame is a plane passing through all three US fiducial markers, and the CT coordinate frame is a plane passing through all three CT fiducial markers. In another embodiment, there are more than three, e.g. four, five, or more, US fiducial markers and the same number of corresponding CT fiducial markers. The US coordinate frame is a plane that passes through or best fits all the US fiducial markers, and the CT coordinate frame is a plane that passes through or best fits all the CT fiducial markers. For example, as the CT coordinate frame is defined based on the three or more CT fiducial markers in the 3D-CT image representation 120, one or more of the three or more CT fiducial markers may reside outside of the CT coordinate frame since a plane can be defined by any three CT fiducial markers.

Each of the US and CT coordinate frames includes a reference origin, three orthogonal axes to represent a 3D space, wherein one of the three orthogonal axes is a normal axis perpendicular to the coordinate frame or plane. Additionally, each reference origin may be coincident along the respective normal axis.

The first image registration operation 400 further includes a step 414 of aligning the US and CT coordinate frames to thereby determine the rigid transformation matrix. Said aligning is based on correspondence of the fiducial markers between the 2D-US image representation 116 and 3D-CT image representation 120. As the fiducial markers may mark vessel bifurcations for easier identification, correspondence between the fiducial markers can be more easily found. Optionally, there is a step 416 of verifying if the alignment is acceptable. Specifically, the step 416 verifies if the correspondence between the fiducial markers are acceptable. If the alignment is not acceptable, such as if one pair of US and CT fiducial markers are not at the same position around the lesion position, the steps 406 and/or 408 are repeated. Accordingly, the steps 406 and/or 408 may be repeated such that the US and CT fiducial markers are arbitrarily defined in an interactive manner. Improved accuracy can thus be achieved via the step 416 by refining or fine-tuning the fiducial markers until the alignment is acceptable.

If the alignment is acceptable, the step 416 proceeds to a step 418 of determining a set of rigid geometric transformations based on alignment of the 2D-US image representation 116 and 3D-CT image representation 120. Specifically, the set of rigid geometric transformations is determined based on alignment of the US and CT coordinate frames. FIG. 4C illustrates a first aligned image representation 124, which is an example of the alignment of the 2D-US image representation 116 and 3D-CT image representation 120 based on the fiducial markers.

A rigid transformation or isometry is a transformation that preserves lengths or distances between every pair of points. A rigid transformation includes reflections, translations, rotations, and combinations of these three transformations. Optionally, the rigid transformation excludes reflections such that the rigid transformation also preserves orientation. In many embodiments, the rigid transformation matrix is determined based on the set of rigid geometric transformations which includes rotations and/or translations. Specifically, the rotations are defined as angular rotations of the normal axes of the US and CT coordinate frames about the three orthogonal axes, and the translations are defined as linear translations between the reference origins of the US and CT coordinate frames along the three orthogonal axes. The rotations and/or translations about/along the three orthogonal axes thus represent up to six degrees of freedom which refers to the freedom of movement of a rigid body in a 3D space. Furthermore, each of the rotations and/or translations is associated with a dimensional parameter, such as angles for the rotations and distances for the translations. The set of rigid geometric transformations is determined based on an ideal alignment of the 2D-US image representation 116 and 3D-CT image representation 120, or more specifically the US and CT coordinate frames, such that the reference origins are coincident, and the normal axes are collinear.

The first image registration operation 400 further includes a step 420 of performing said determining of the rigid transformation matrix based on the set of rigid geometric transformations. Points or positions on one of the 2D-US image representation 116 and 3D-CT image representation 120 are transformable to the other via the rigid transformation matrix. For example, a voxel in the 3D-CT image representation 120 is firstly defined in the CT coordinate frame. The voxel is then transformed to the US coordinate frame via the rigid transformation matrix, thereby localizing the voxel in the US coordinate frame. The voxel may represent the lesion position in the 3D-CT image representation 120, and the lesion can thus be localized relative to the 2D-US image representation, or more specifically in the US coordinate frame, via the rigid transformation matrix.

In some embodiments, the US coordinate frame is identical to the reference coordinate frame of the transducer probe 114/reference position sensor 118. In some other embodiments, the US coordinate frame differs from the reference coordinate frame by a reference rigid transformation. Accordingly, points or positions in the US coordinate frame/CT coordinate frame are transformable to the reference coordinate frame, such that the positions are localized in the reference coordinate frame and positioned relative to the transducer probe 114/reference position sensor 118.

The rigid transformation matrix represents an initial alignment of the 2D-US image representation 116 and 3D-CT image representation 120. However, as the US and CT fiducial markers are arbitrarily defined, the 2D-US image representation 116 and 3D-CT image representation 120, or more specifically the US and CT coordinate frames, may not be properly aligned. For example, the reference origins may be coincident, but the normal axes may not be collinear, or vice versa. The rigid transformation matrix determined in the first image registration operation 400 is thus subjected to the second image registration operation 500 for refining the rigid transformation matrix.

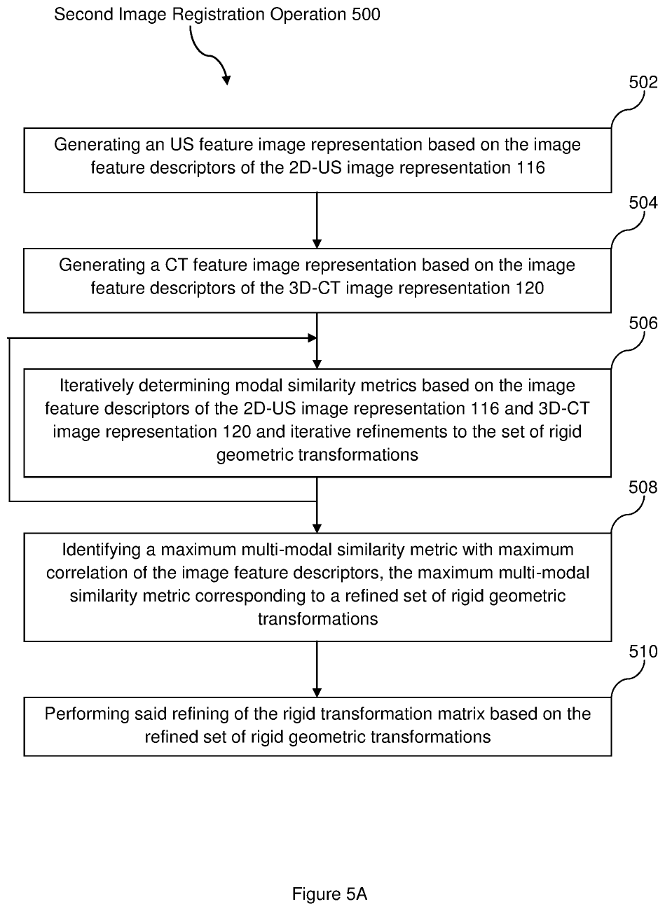

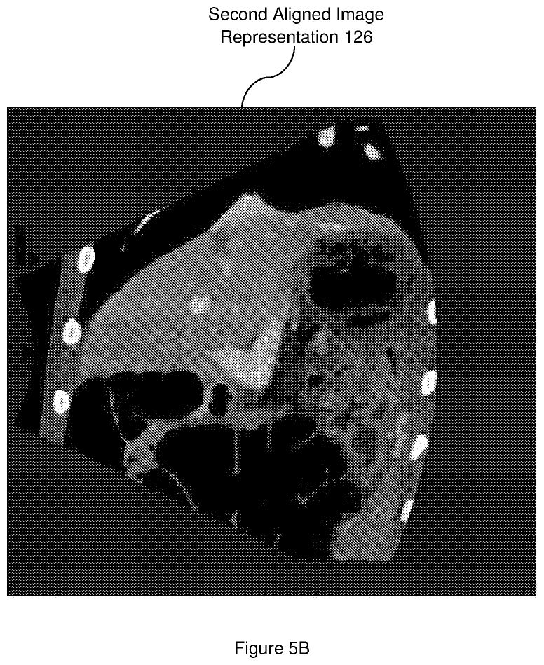

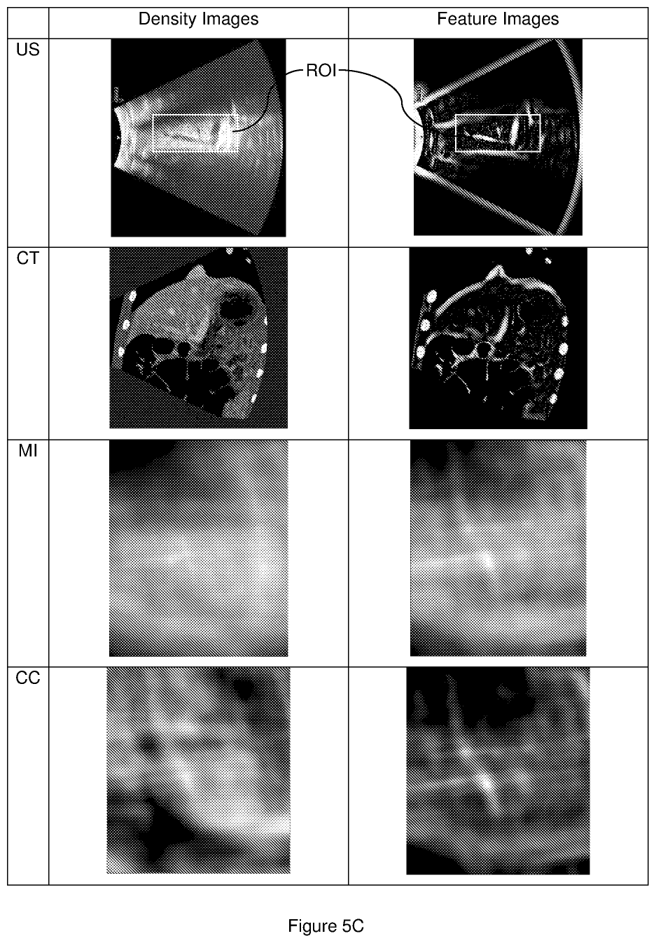

With reference to FIG. 5A, the second image registration operation 500 includes a step 502 of generating an US feature image representation based on the image feature descriptors of the 2D-US image representation 116, and a step 504 of generating a CT feature image representation based on the image feature descriptors of the 3D-CT image representation 120. It will be appreciated that the steps 502 and 504 may be performed in any sequence or simultaneously.

The image feature descriptors are based on composite features of vascular tissues extracted from the 2D-US image representation 116 and 3D-CT image representation 120. For example, the organ is a liver and the vascular tissues include the hepatic vessels. The composite features of the hepatic vessels describe their properties of density and local shape/structure. The density feature is the relative density of the hepatic vessels estimated with a Gaussian mixture model. The local shape feature of the hepatic vessels is measured with a 3D Hessian matrix or matrix-based filter. The density feature is estimated from the 2D-US image representation 116 and 3D-CT image representation 120 of the liver, and the local shape feature is measured using eigenvalues of the 3D Hessian matrix (References [25] and [26]).

The local shape feature at a voxel of the 3D-CT image representation 120 is calculated as:

$$LS = \{ \begin{matrix}\vert {λ}_{2} \vert - {λ}_{1} , & if \vert {λ}_{1} \vert \leq \vert {λ}_{2} \vert \leq \vert {λ}_{3} \vert , and {λ}_{2} , {λ}_{3} < 0 \\ 0 & otherwise\end{matrix}$$

λ₁, λ₂, and λ₃ are the eigenvalues of the 3D Hessian matrix at that voxel, with the absolute values in ascending order. A multi-scale filtering scheme is adopted to tackle hepatic vessels of various sizes. The multi-scale filtering scheme works by smoothing the 3D-CT image representation 120 using a Gaussian filter with various kernel sizes before Hessian filtering. The kernel sizes are set as 1, 3, 5, and 7 mm. The maximum value among single-scale filter responses is retained as local shape feature at this voxel. For each pixel of the 2D-US image representation 116 and voxel of the 3D-CT image representation 120, the image feature descriptor of the pixel/voxel predicts its probability to be or include hepatic vessels.

In some embodiments, the US and CT feature image representations are generated using a supervised learning-based method or framework, such as a Support Vector Classifier (SVC), employed by the image registration module 108. In the steps 502 and 504, the composite features of the hepatic vessels in the 2D-US image representation 116 and 3D-CT image representation 120 of the liver are extracted using the SVC, and the US and CT feature image representations are generated using the image feature descriptors of the composite features.

The image registration module 108 may be trained using training data from a set of training images for segmentation of the vascular tissues, specifically the hepatic vessels, and for determining the image feature descriptors to generate the US and CT feature image representations. The training images may be selected to include those that represent the vascular tissues/hepatic vessels. The training data includes the density and local shape features of the vascular tissues/hepatic vessels in the training images. The training data is then input to the SVC to train the image registration module 108.

The second image registration operation 500 further includes a step 506 of iteratively determining modal similarity metrics based on the image feature descriptors of the 2D-US image representation 116 and 3D-CT image representation 120 and iterative refinements to the set of rigid geometric transformations. Specifically, each iteration of determining a modal similarity metric is performed based on the US and CT feature image representations and an iteration of the iterative refinements to the rigid geometric transformations. The iterative refinements are based on adjustments in one or more of the degrees of freedom, i.e. any number from one to six degrees of freedom, to refine or fine-tune the dimensional parameters associated with the rotations/translations.

The second image registration operation 500 further includes a step 508 of identifying a maximum multi-modal similarity metric with maximum correlation of the image feature descriptors, the maximum multi-modal similarity metric corresponding to a refined set of rigid geometric transformations. Specifically, the maximum multi-modal similarity is associated with maximum correlation of the US and CT feature image representations. The maximum correlation is determined using a convergent iterative method, such as a gradient descent algorithm.

Accordingly, in the step 506, refinements to the rigid geometric transformations are made iteratively and a multi-modal similarity metric is determined for each iteration of refinements. The iterative refinements lead to convergence of the multi-modal similarity metrics to the maximum multi-modal similarity metric. More iterations of the refinements would lead the multi-modal similarity metrics closer to the maximum. The refined set of rigid geometric transformations is determined based on the final iteration of refinements.

The second image registration operation 500 further includes a step 510 of performing said refining of the rigid transformation matrix based on the refined set of rigid geometric transformations. FIG. 5B illustrates a second aligned image representation 126, which is an example of the refined alignment of the 2D-US image representation 116 and 3D-CT image representation 120 based on the feature image representations and the refined set of rigid geometric transformations.

In some embodiments with reference to FIG. 5C, the multi-modal similarity metrics are determined using mutual information (MI) and correlation coefficient (CC) measurements. For purpose of comparison, the multi-modal similarity metrics are determined based on the original image representations, i.e. the 2D-US image representation 116 and 3D-CT image representation 120 after the first image registration operation 400, and based on the corresponding US and CT feature image representations. The original image representations are compared based on density features, and the feature image representations are compared based on the composite features. Furthermore, in the 2D-US image representations 116, a region of interest (ROI) is identified for determining the multi-modal similarity metrics. The ROI includes local vascular information such as vessel bifurcations.

FIG. 5C illustrates the MI and CC measurements of the maximum multi-modal similarity metrics associated with the refined alignment. In both MI and CC measurements, the higher the intensity, the higher the similarity. The regions with the highest intensity or the brightest regions indicate the local/global maxima. For the original image representations, the refined alignment resulted in local maximum for both MI and CC measurements. The original image representations may not be reliability used for comparing images using such multi-modal similarity metrics. On the other hand, for the feature image representations, the refined alignment resulted in global maximum for both MI and CC measurements. Notably, in the CC measurement, there is one isolated maximum peak which is the global maximum selected for determining the maximum multi-modal similarity metric.

The second image registration operation 500 thus refines the rigid transformation matrix that improves alignment of the 2D-US image representation 116 and 3D-CT image representation 120. Points or positions on one of the 2D-US image representation 116 and 3D-CT image representation 120 are transformable to the other via the refined rigid transformation matrix, and consequently transformable to the reference coordinate frame, such that the positions are localized in the reference coordinate frame and positioned relative to the transducer probe 114/reference position sensor 118.

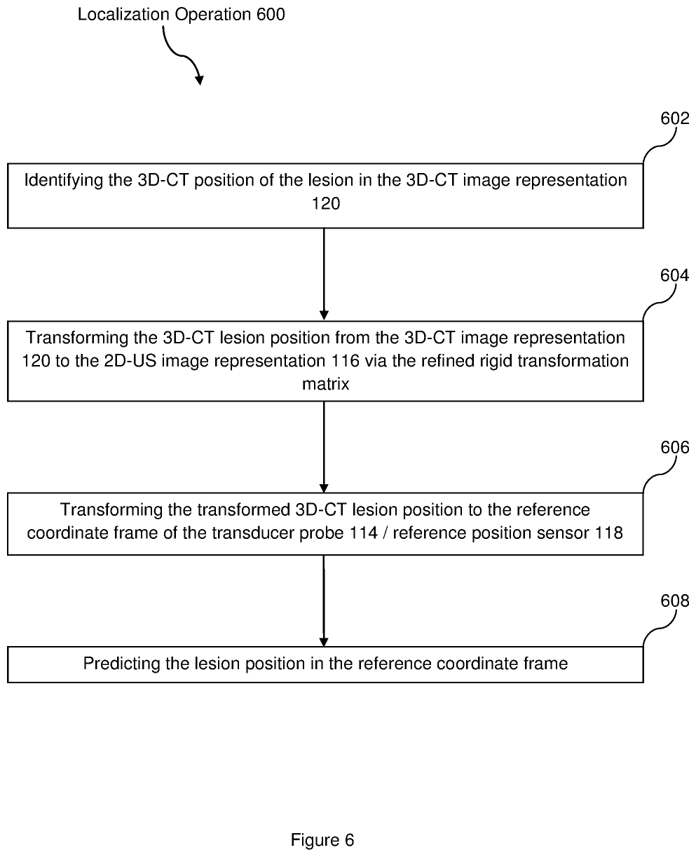

With reference to FIG. 6, the localization operation 600 includes a step 602 of identifying the 3D-CT position of the lesion in the 3D-CT image representation 120. The 3D-CT lesion position is transformed to the reference coordinate frame based on the refined rigid transformation matrix, thereby localizing the lesion in the reference coordinate frame and positioning the lesion relative to the transducer probe 114/reference position sensor 118. Specifically, the localization operation 600 includes a step 604 of transforming the 3D-CT lesion position from the 3D-CT image representation 120 to the 2D-US image representation 116 via the refined rigid transformation matrix. The localization operation 600 further includes a step 606 of transforming the transformed 3D-CT lesion position to the reference coordinate frame of the transducer probe 114/reference position sensor 118. In one embodiment, the reference coordinate frame is identical to the US coordinate frame of the 2D-US image representation 116. In another embodiment, the US coordinate frame differs from the reference coordinate frame by a reference rigid transformation. The localization operation 600 further includes a step 608 of predicting the lesion position in the reference coordinate frame.

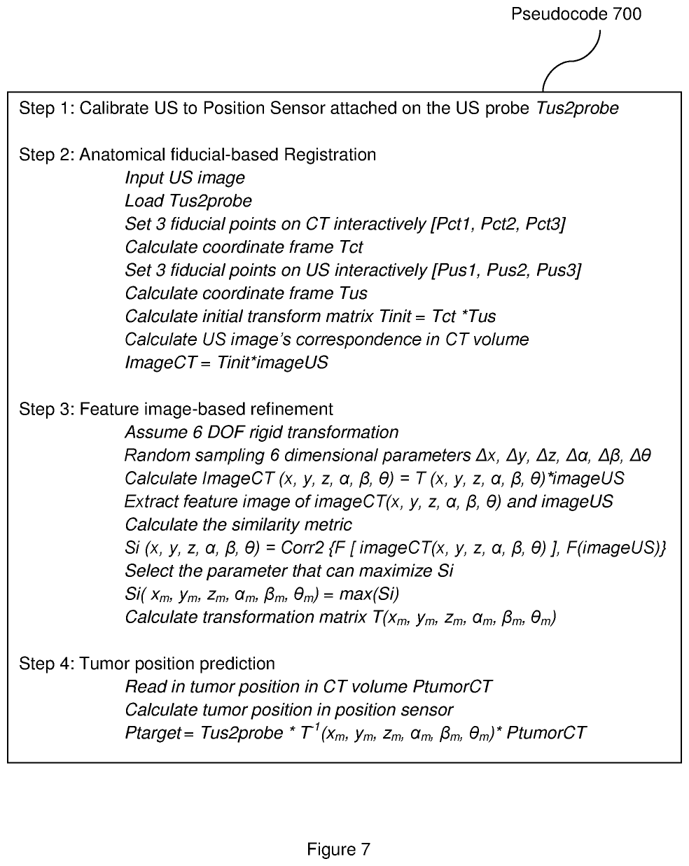

Advantageously, the method 200 improves localization of a lesion in an organ of a subject, such as a liver tumor, using the 2D-US image representation 116 and 3D-CT image representation 120 of the organ, as well as refinements to the rigid transformation matrix for alignment of the 2D-US image representation 116 and 3D-CT image representation 120. An example of a pseudocode 700 for the method 200 is shown in FIG. 7.

An experimental study was conducted to evaluate the performance of the method 200 for localizing a lesion in an organ. The method 200 was performed for localizing a target tumor in a liver of a pig during a respiration cycle. 2D-US image representations 116 of the liver were acquired using the transducer probe 114 on the same position of the pig, including a 2D-US image representation 116a at the end of the inhalation phase and a 2D-US image representation 116b at the end of the exhalation phase. 3D-CT image representations 120 of the liver were pre-acquired from the same pig at the same position, including a 3D-CT image representation 120a at the end of the inhalation phase and a 3D-CT image representation 120b at the end of the exhalation phase.

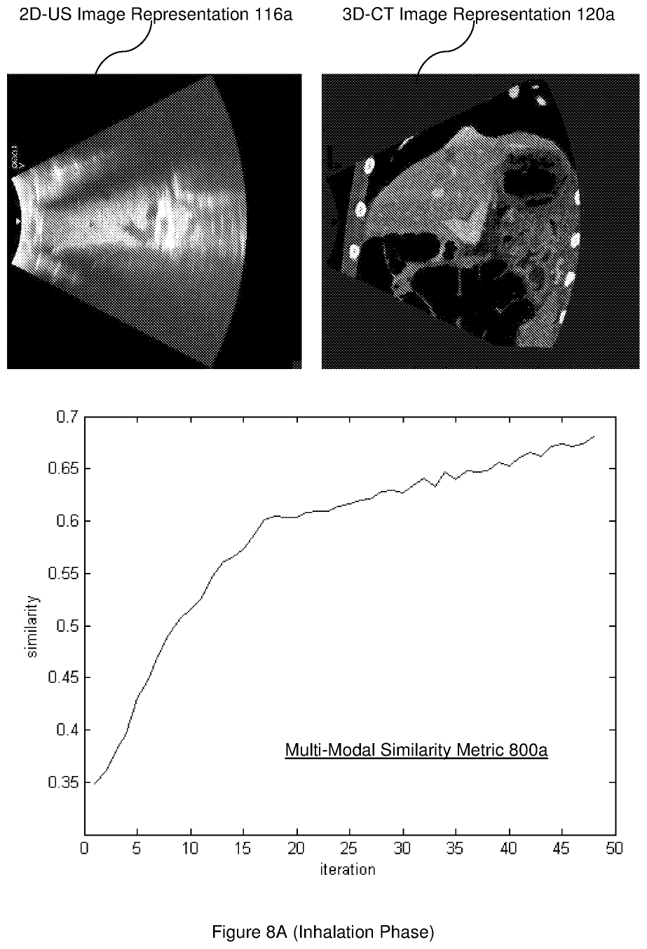

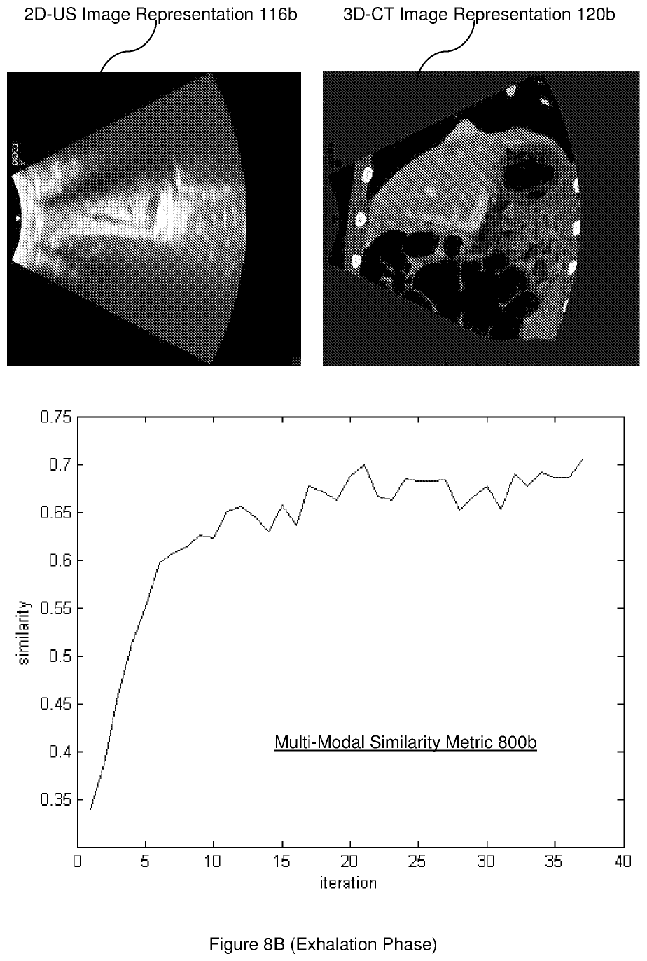

The first image registration operation 400 and second image registration operation 500 in the method 200 registers the 2D-US and 3D-CT image representations 116a and 120a at the end of the inhalation phase, and registers the 2D-US and 3D-CT image representations 116b and 120b at the end of the exhalation phase. With reference to FIG. 8A (inhalation phase) and FIG. 8B (exhalation phase), at the refined alignment of the 2D-US and 3D-CT image representations 116a-120a and 116b-120b, the multi-modal similarity metrics 800a and 800b approached and converged to the global maximums for both inhalation and exhalation phases after dozens of iterations of refining the rigid transformation matrix. The refined rigid transformation matrix was determined based on the global maximums of the multi-modal similarity metrics.

The fiducial registration error (FRE) and target registration error (TRE) were measured and used for evaluating the method 200. The FRE is the root mean square distance among the fiducial markers after image registration based on the refined rigid transformation matrix. In the first image registration operation 400, three fiducial markers were defined around the lesion positions in each of the 2D-US and 3D-CT image representations 116ab and 120ab. The fiducial markers marked the portal vein bifurcations around the lesion, and the FRE was calculated to be 1.24 mm.

The TRE is the root mean square error in position change estimation. A common target point was first selected in the 3D-CT image representations 120a and 120b. Corresponding coordinates were determined in the 2D-US image representations 116a and 116b based on the refined rigid transformation matrix. The CT coordinates change of the target points in the 3D-CT image representations 120a and 120b showed how the liver moved during the respiration cycle, and was viewed as the ground truth. Similarly, the US coordinates change in the 2D-US image representations 116a and 116b showed how the liver moved during the same respiration cycle. The CT coordinates change was calculated to be −0.7 mm, −11.4 mm, and 4.1 mm in three orthogonal axes, respectively. The US coordinates change was calculated to be 4.5 mm, −5.5 mm, and 2.2 mm in the same three orthogonal axes, respectively. The TRE was calculated to be 8.02 mm.

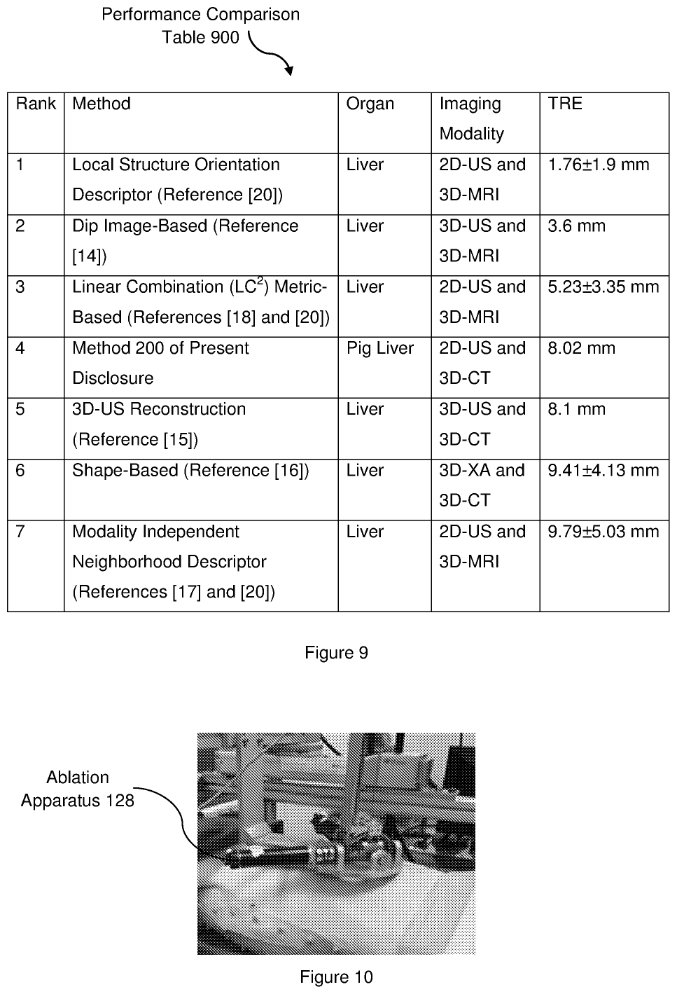

FIG. 9 illustrates a performance comparison table 900 comparing the performance of the method 200 with other known methods. Notably, the method 200 is similar to the performance of a 3D-US and 3D-CT image registration.

The system 100 and method 200 may be used in collaboration in an IGI for treating the lesion, such as RFA. In some embodiments, the system 100 includes an ablation apparatus 128 for RFA of the lesion. An example of the ablation apparatus 128 is illustrated in FIG. 10. The ablation apparatus 128 includes a RFA probe for insertion into the lesion and a set of position sensors calibrated with the RFA probe. The RFA probe generates radiofrequency waves to increase the temperature within the lesion, resulting in ablation, or ideally destruction, of the lesion. The set of position sensors of the ablation apparatus 128 may be part of a robotic system that controls and actuates the RFA probe to target the lesion. The RFA may be guided by the transducer probe 114 to localize the lesion relative to the transducer probe 114/reference position sensor 118. The set of position sensors of the ablation apparatus 128 may be cooperative with the transducer probe 114/reference position sensor 118, such that the RFA probe can be positioned in the reference coordinate frame of transducer probe 114/reference position sensor 118 for ultrasonically guiding the RFA probe to the localized lesion in the reference coordinate frame. Accordingly, the method 200 is able to assist a robotic intervention procedure to improve targeting of the lesion for more effective ablation thereof.

Embodiments of the present disclosure describe a system 100 and method 200 for localizing a lesion in an organ of a subject. The method 200 uses a two-stage image registration process to register the 2D-US image representation 116 and 3D-CT image representation 120. The two-stage image registration process includes the first stage 204 (first image registration operation 400) and second stage 206 (second image registration operation 500). The first image registration operation 400 is based on fiducial markers and may be referred to as a fiducial-based registration, and the second image registration operation 500 is based on image feature descriptors and may be referred to as a feature-based registration. The initial rigid transformation matrix is determined by alignment of the fiducial markers in the 2D-US image representation 116 and 3D-CT image representation 120. The initial rigid transformation matrix is then refined by searching for the maximum correlation of the two US and CT feature image representations using a supervised learning-based method or framework and a convergent iterative method, such as the gradient descent algorithm. After the two-stage image 2D-US image representation 116/3D-CT image representation 120 coordinate frame are transformable to the reference coordinate frame via the refined rigid transformation matrix, such that the positions are localized in the reference coordinate frame and positioned relative to the transducer probe 114/reference position sensor 118. Localization of the lesion using the method 200 does not require 3D reconstruction from a series of 2D-US image scans or simulation from 3D-CT image volumes, and does not conduct global organ registration which lowers computational complexity. The method 200 may be used in collaboration with an IGI such as US-guided RFA to improve localization and targeting of the lesion for more effective ablation. As shown in the performance comparison table 900 in FIG. 9, the performance of the method 200 is encouraging and addresses various disadvantages of other known methods.

In the foregoing detailed description, embodiments of the present disclosure in relation to system and computerized method for localizing a lesion in an organ of a subject using 2D-US and 3D-CT image representations of the organ are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

### REFERENCES

[1] U.S. Pat. No. 8,942,455—2D/3D image registration method.
[2] U.S. Pat. No. 8,457,373—System and method for robust 2D-3D image registration.
[3] U.S. Pat. No. 7,940,999—System and method for learning-based 2D/3D rigid registration for image-guided surgery using Jensen-Shannon divergence.
[4] U.S. Pat. No. 9,135,706—Features-based 2D-3D image registration.
[5] U.S. Pat. No. 9,262,830—2D/3D image registration.
[6] U.S. Pat. No. 8,675,935—Fast 3D-2D image registration method with application to continuously guided endoscopy.
[7] United States Patent Publication 20090161931—Image registration system and method.
[8] United States Patent Publication 20150201910—2D-3D rigid registration method to compensate for organ motion during an interventional procedure.
[9] U.S. Pat. No. 9,521,994—System and method for image guided prostate cancer needle biopsy.
[10] United States Patent Publication 20140193053—System and method for automated initialization and registration of navigation system.
[11] United States Patent Publication 20160078633—Method and system for mesh segmentation and mesh registration.
[12] United States Patent Publication 20120253200—Low-cost image-guided navigation and intervention systems using cooperative sets of local sensors.
[13] United States Patent Publication 20170243349—Automatic region-of-interest segmentation and registration of dynamic contrast-enhanced images of colorectal tumors.
[14] International Patent Publication WO2015173668—Reconstruction-free automatic multi-modality ultrasound registration.
[15]G. P. Penney, J. M. Blackall, M. S. Hamady, T. Sabharwal, A. Adam, D. J. Hawkes, “Registration of freehand 3D ultrasound and magnetic resonance liver images”, Medical Image Analysis, 8(2004): 81-91.
[16] W. Wein, S. Brunke, A. Khamene, M. R. Callstrom, N. Navab, “Automatic CT-ultrasound registration for diagnostic imaging and image-guided intervention”, Medical Image Analysis, 12(2008): 577-585.
[17] N. Subramanian, E. Pichon, S. B. Solomon, “Automatic registration using implicit shape representations: application in intraoperative 3D rotational angiography to preoperative CTA registration”, Int J CARS, 4(2009): 141-146.
[18] M. P. Heinrich, M. Jenkinson, M. Bhushan, T. Matin, F. V. Gleeson, S. M. Brady, J. A. Schnabel, “MIND: Modality independent neighborhood descriptor for multi-modal deformable registration”, Medical Image Analysis, 16(2012): 1423-1435.
[19] B. Fuerst, W. Wein, M. Muller, N. Navab, “Automatic ultrasound-MRI registration for neurosurgery using the 2D and 3D LC2 metric”, Medical Image Analysis, 18(2014): 1312-1319.
[20] M. Yang, H. Ding, L. Zhu, G. Wang, “Ultrasound fusion image error correction using subject-specific liver motion model and automatic image registration”, Computers in Biology and Medicine, 79 (2016): 99-109.
[21]M. Yang, H. Ding, J. Kang, L. Cong, L. Zhu, G. Wang, “Local structure orientation descriptor based on intra-image similarity for multimodal registration of liver ultrasound and MR images”, Computers in Biology and Medicine, 76(2016): 69-79.
[22] J. Jiang, S. Zheng, A. W. Toga and Z. Tu, “Learning based coarse-to-fine image registration,” 2008 IEEE Conference on Computer Vision and Pattern Recognition, (2008):1-7.
[23] R. W. K. So, A. C. S. Chung, “A novel learning-based dissimilarity metric for rigid and non-rigid medical image registration by using Bhattacharyya Distances,” Pattern Recognition, 62(2017):161-174.
[24] G. Wu, F. Qi, D. Shen, “Learning-based deformable registration of MR Brain images,” IEEE Trans. Medical Imaging, 25(9)(2006):1145-1157.
[25]Y. Chi, W. Huang, J. Zhou, L. Zhong, S. Y. Tan, F. Keng, S. Low, R. Tan, “A Composite of Features for Learning-Based Coronary Artery Segmentation on Cardiac CT Angiography”, Proceedings of MLMI 2015, 6th International Workshop Held in Conjunction with MICCAI 2015, Munich, 9352 (2015): 271-279.
[26]Y. Chi, J. Liu J, S. K. Venkatesh, S. Huang, J. Zhou, Q. Tian, W. L. Nowinski, “Segmentation of liver vasculature from contrast enhanced CT images using context-based voting,” IEEE Trans Biomed Eng. 58(8)(2011): 2144-2153.

1. A computerized method for localizing a lesion in an organ of a subject, the method comprising performing:
a first image registration operation for determining a rigid transformation matrix based on alignment of a two-dimensional ultrasound (2D-US) image representation and a three-dimensional computed tomography (3D-CT) image representation of the organ, the 2D-US image representation acquired from a transducer probe;
a second image registration operation for refining the rigid transformation matrix based on image feature descriptors of the 2D-US and 3D-CT image representations; and
a localization operation for localizing the lesion relative to the transducer probe based on the refined rigid transformation matrix and a 3D-CT position of the lesion in the 3D-CT image representation.

2. The method according to claim 1, the method further comprising performing, before the first image registration operation, a calibration operation for calibrating the transducer probe.

3. The method according to claim 2, the calibration operation comprising defining a reference coordinate frame of the transducer probe, wherein the lesion is localized in the reference coordinate frame.

4. The method according to claim 1, the first image registration operation comprising:
receiving the 2D-US image representation acquired from the transducer probe used on the subject; and
retrieving, from an image database, the 3D-CT image representation pre-acquired from the subject.

5. The method according to claim 1, the first image registration operation comprising:
defining three or more CT fiducial markers around the 3D-CT lesion position in the 3D-CT image representation; and
defining three or more US fiducial markers in the 2D-US image representation corresponding to the CT fiducial markers.

6. The method according to claim 5, the first image registration operation further comprising:
defining a CT coordinate frame based on the CT fiducial markers;
defining a US coordinate frame based on the US fiducial markers; and
aligning the US and CT coordinate frames to thereby determine the rigid transformation matrix.

7. The method according to claim 1, the first image registration operation comprising:
determining a set of rigid geometric transformations based on alignment of the 2D-US and 3D-CT image representations; and
performing said determining of the rigid transformation matrix based on the set of rigid geometric transformations.

8. The method according to claim 7, wherein the set of rigid geometric transformations comprises rotations and/or translations in up to six degrees of freedom.

9. The method according to claim 7, the second image registration operation comprising iteratively determining modal similarity metrics based on the image feature descriptors of the 2D-US and 3D-CT image representations and iterative refinements to the set of rigid geometric transformations.

10. The method according to claim 9, wherein the iterative refinements are based on one or more of the degrees of freedom.

11. The method according to claim 9, the second image registration operation further comprising identifying a maximum multi-modal similarity metric associated with maximum correlation of the image feature descriptors, the maximum multi-modal similarity metric corresponding to a refined set of rigid geometric transformations.

12. The method according to claim 11, wherein the maximum correlation of the image feature descriptors is determined using a gradient descent algorithm.

13. The method according to claim 11, the second image registration operation further comprising performing said refining of the rigid transformation matrix based on the refined set of rigid geometric transformations.

14. A system for localizing a lesion in an organ of a subject, the system comprising:
a transducer probe for acquiring a two-dimensional ultrasound (2D-US) image representation of the organ; and
a computer device communicable with the transducer probe, the computer device comprising:
an image registration module configured for performing:
a first image registration operation for determining a rigid transformation matrix based on alignment of the 2D-US image representation and a three-dimensional computed tomography (3D-CT) image representation of the organ; and
a second image registration operation for refining the rigid transformation matrix based on image feature descriptors of the 2D-US and 3D-CT image representations; and
a localization module configured for performing a localization operation for localizing the lesion relative to the transducer probe based on the refined rigid transformation matrix and a 3D-CT position of the lesion in the 3D-CT image representation.

15. The system according to claim 14, further comprising a calibration module configured for performing a calibration operation for calibrating the transducer probe.

16. The system according to claim 14, further comprising a reference position sensor disposed on the transducer probe, wherein the lesion is localized relative to the reference position sensor.

17. The system according to claim 16, further comprising an ablation apparatus for radio frequency ablation (RFA) of the lesion.

18. The system according to claim 17, the ablation apparatus comprising a RFA probe for insertion into the lesion and a set of position sensors calibrated with the RFA probe.

19. The system according to claim 18, wherein the reference position sensor is cooperative with the set of position sensors for ultrasonically guiding the RFA probe to the localized lesion.

20. The system according to claim 14, wherein the image registration module is trained using training data from a set of training images for determining the image feature descriptors.
